(19) United States
(12) Patent Application Publication
(10) Pub. No.: US 2025/0380879 A1
(43) Pub. Date: Dec. 18, 2025

(54) SYSTEM AND METHOD FOR SENSORS INTEGRATION FOR NON-STATIC CONTINUOUS BLOOD PRESSURE MONITORING

(71) Applicant: LIVEMETRIC (MEDICAL) S.A, Luxembourg (LU)

(72) Inventors: Mari´a Irene DE ORBE IZQUIERDO, Luxembourg (LU); Ori HAY, Haifa (IL)

(21) Appl. No.: 19/105,934

(22) PCT Filed: Aug. 31, 2023
(86) PCT No.: PCT/IL2023/050934

§ 371 Date: Feb. 24, 2025

Related U.S. Application Data
(60) Provisional application No. 63/403,757, filed on Sep. 4, 2022.

(51) Int. Cl. A61B 5/021; A61B 5/00; A61B 5/024; A61B 5/0245

(52) U.S. Cl. CPC A61B 5/021; A61B 5/02416; A61B 5/0245; A61B 5/681; A61B 5/721; A61B 5/7221; A61B 5/7246; A61B 5/7264; A61B 2560/0462; A61B 2562/0219; A61B 2562/0247

(57) ABSTRACT

There are provided herein methods and systems for continuous blood pressure measurement of a non-static subject. The systems and methods utilize fusion of blood pressure related data, pulse-related data and motion related signals obtained from corresponding sensors, to compensate for motion artifacts and accurately determine and monitor blood pressure during non-static periods.

### FIELD OF THE INVENTION

The present disclosure relates generally to system, device, and methods for non-static continuous blood pressure monitoring utilizing integration of various sensors.

### BACKGROUND

High blood pressure is a common condition in which the long-term force of the blood against the artery walls is high enough that it may eventually cause health problems, such as heart disease. Blood pressure (BP) is determined both by the amount of blood the heart pumps and the amount of resistance to blood flow in the arteries. The more blood the heart pumps and the narrower the arteries are, the higher the blood pressure is. During each heartbeat, the blood pressure varies between a maximum (i.e., systolic) and a minimum (i.e., diastolic) pressure.

Wearable devices for monitoring blood pressure and pulse rate have difficulties providing measurement while the subject is non-static (engaged in activity). Motion due to subjects' activity induces artifacts and noise that cause difficulties to measure or even prevents measurement of blood pressure and pulse rate for wearable blood pressure monitors. Subjects' motion during activities disrupts the ability of blood pressure monitors to perform measurements and consequently affects their accuracy and effectiveness. Motion artifacts and related noise usually reduce the accuracy of blood pressure measurement or even prohibit measuring due to poor signal quality because of excessive motion artifacts.

Traditional blood pressure measurements require inflatable cuffs, which are gradually deflated from a state of full vessel occlusion to a lower pressure while listening using a mechanical sensor (e.g., stethoscope) to the sounds generated by the blood flow eddies in the vessel. An advantage of this method is its relative robustness to arm motion, while a disadvantage is its large form factor and the need for either manual inflation by the user or an automatic pump, which requires large quantities of energy, and such technology thus cannot be used in wearable devices.

Other technologies for noninvasive blood pressure measurement include: non-contact sensors-such as optical PPG (PhotoPlethysmoGraphy), RF (radio frequency), or ultrasound, applanation tonometry using surface pressure sensors, or a combination of multiple sensors such as PPG with ECG (ElectroCardioGraphy) sensors to compute BP based on various techniques. The former group of non-contact sensors are commonly proposed as non-invasive sensors for blood pressure measurement for wearable devices because of the comfort of their non-contact property. However, these sensors don't measure pressure directly but measure characteristics such as blood volume or blood flow and calculate blood pressure using techniques such as PPT (Pulse Transit Time) or PWV (Pulse Wave Velocity) that also require calibration.

Usually wearable medical devices, in order to assess motion or activity, also incorporate one or more additional motion sensors such as, but not limited to accelerometer, gyroscope, magnetometer, or other form of inertial measurement unit (IMU) sensors.

As stated above, motion affects the accuracy and efficacy of wearable devices. The majority of wearable devices which are based on optical sensors (PPG) or ECG, use common methods for motion cancelation. However, for blood pressure monitors or devices including pressure sensors, such motion compensation is hardly ever implemented. Pressure sensors in general and particularly those housed with wearable devices, are very sensitive to motion because they directly measure mechanical properties—and not optical (PPG) or electrical (ECG). Motion induced artifacts and noise cause difficulties in measurement of blood pressure and pulse rate for wearable blood pressure monitors regardless of the technology used for measurement.

Blood pressure calculation using a wearable device and more specifically hand worn device relies on adequate signal quality. Most of these sensors are sensitive to motion, causing lower signal-to-noise ratio (SNR) and motion artifacts that either reduce the device accuracy or completely prevent any measurement. Motion artifacts are considered external sources of quality degradation of the measured data due to the body movement, such as arm motion, muscle tremor or shivering. In particular, when monitoring continuously the BP in the wrist while the subject is non-static, not only the sudden movements need to be considered, but also those related to the daily activities, where the artifact remains over time. The inaccuracy caused by the former may be punctual and easier to handle, while the latter may corrupt the signal down to the point of missing any track of BP values. To overcome these problems, motion sensing sensors to detect or avoid motion while measuring blood pressure, or partial motion cancelation or compensation solely based on blood pressure and motion sensors have been proposed. For example, U.S. Pat. No. 6,176,831 is directed to an apparatus and method for non-invasively monitoring a subject's arterial blood pressure. For example, U.S. Pat. No. 7,429,245 is directed to Motion management in a fast blood pressure measurement device. For example, CN107212858 patent application is directed to Physiological information collection device and method based on exercise state. For example, US Patent No. U.S. Pat. No. 8,475,370 is directed to a method for measuring patient motion, activity level, and posture along with PTT-based blood pressure.

Nevertheless, there is a need in the art for devices and methods for providing continuous blood pressure monitoring for non-static users using wearable devices, in an accurate, cost effective and reliable manner, by utilizing fusion of information from various related sensors.

### SUMMARY

According to some embodiments, there are provided herein systems, devices and methods for continuous, non-invasive monitoring of vital signs (such as blood pressure) of a non-static subject. In some embodiments, the systems, devices and methods disclosed herein allow non-invasive continuous blood pressure measurement while the subject is engaged in activity (i.e., non-static), utilizing corresponding wearable device, which is configured to provide accurate blood pressure monitoring, based on integration of data from a plurality of sensors.

According to some embodiments, monitoring of blood pressure using a wearable device involves streamed measurement of physiological signals and continuous calculation of BP and pulse rate values, which requires heartbeats separation as a vital step of the calculation process, because Systolic and Diastolic blood pressure values are defined based on heartbeat waveform. Therefore, measurement of Systolic and Diastolic BP values from pulse waveform in general and continuous blood pressure measurement in particular are inherently calculated per heartbeat (single heartbeat or over several heartbeats) to capture the dynamical nature of blood pressure. Motion artifacts often introduce patterns and noise into the pulse waveform, making the detection and separation of heartbeats much harder, and cumbersome. Accordingly, the advantageous methods disclosed herein allow motion compensation and accurate detection of heartbeat onsets, based on fusion of information from various related sensors.

According to some embodiments, the systems, devices and methods disclosed herein enable non static continuous vital signs monitoring using wearable devices and utilizing pressure sensors. Such systems, devices and methods overcome motion induced artifacts and enable continuous measurement, by incorporating one or more additional sensors and integrating the information from various sensors. Thus, in some embodiments, at least some of the additional sensors are capable of producing hemodynamical pulse-related signals capable of pulse (heartbeat) detection in the presence of motion, such as, but not limited to, PPG and ECG. The use of such sensors, which are more robust to motion, can be used to verify that the data used for determining blood pressure, heart rate, and the like, is correct and not caused by measuring motion induced artifacts (for example, artifacts resembling heartbeat).

According to some embodiments, the device and/or method may be configured to record blood pressure waveforms and analyze the changes in the shape of the waveform.

Advantageously, the methods and devices disclosed herein enable continuous blood pressure measurements during non-static periods, by mitigating motion-related artifacts that may otherwise affect the blood pressure measurement.

In some embodiments, the disclosed methods which utilize fusion of data obtained from various sensors to reduce or cancel motion induced artifacts and improve signal quality, can advantageously provide more accurate blood pressure measurement and improve the ratio of successful measurements (not discarded due to poor signal quality), in particular when subjects are non-static.

According to some embodiments, there is provided a wearable blood pressure and vital signs monitoring device and system, which may include a plurality of sensors and a processing unit configured to apply a method for sensors' data fusion enabling static and non-static continuous monitoring of vital signs, providing accurate vital signs measurements while the subject is non-static, e.g., during activity. In some embodiments, such devices and systems are advantageous over currently used blood pressure monitoring devices due to the plurality of sensors used, and the data fusion methods applied. The vital signs monitoring device includes a plurality of sensors which include at least one pressure sensor, at least one additional sensor capable of measuring cardiovascular physiological (pulse related) signals (such as, but not limited to, PPG, ECG, Impedance cardiography (ICG), or phonocardiography), and optionally at least one motion related sensor (such as, but not limited to, accelerometer, gyroscope, magnetometer). The sensor fusion mechanisms and methods disclosed herein overcome motion problems and enable continuous measurement, by incorporating data of physiological sensors in addition to motion related sensors and using data fusion methods, which include, inter alia, algorithms for integration of information/data from the various sensors.

According to some embodiments, there are thus provided methods that enable non static continuous vital signs monitoring utilizing wearable devices having pressure sensors. Such methods overcome motion problems and enable continuous measurement, by incorporating information from additional sensors and integration of the information from the multiple sensors. Thus, in accordance with some embodiments, the device disclosed herein includes or is associated with “pulse-related sensors” that are capable of producing hemodynamical “pulse-related signal”, which are sensors allowing pulse (heartbeat) detection. Such sensors may include, for example, photoplethysmogram (PPG), electrocardiogram (ECG), Impedance cardiography (ICG), phonocardiography (microphones), and the like, or any combination thereof. Such sensors are more robust to motion and information therefrom can thus facilitate ensuring that the data used for determination of blood pressure is correct (and is not attributed to a motion artifact that can resemble, for example, heartbeat).

According to some embodiments, there is provided a method for continuous, non-invasive blood pressure measurement of subject while being active, the method includes:
receiving at least one blood pressure related signal from at least one pressure sensor comprised in a wearable device;
receiving at least one pulse related signal from at least one pulse sensor;
calculating a correlation score between the blood pressure signal and the pulse related signal, said correlation score is indicative of quality and/or validity of the blood pressure signal; and
calculating blood pressure if the correlation score is above a predetermined threshold.

According to some embodiments, the method may further include receiving a motion related signal from at least one motion sensor.

According to some embodiments, the method may further include a step of synchronizing the receiving of the blood pressure signal, the pulse-related signal and/or the motion related signal.

According to some embodiments, the method may further include preprocessing the blood pressure signal and/or the pulse related signal prior to calculating the correlation.

According to some embodiments, the preprocessing may include one or more feature selection and/or onset detection in the blood pressure signal and/or the pulse related signal.

According to some embodiments, the preprocessing may include motion cancelation or motion compensation algorithms applied to the blood pressure signal and/or the pulse related signal.

According to some embodiments, the method may further include the correlation score may be determined using a correlation function, configured to be applied to one or more of the obtained signals, or any parameters derived therefrom.

According to some embodiments, the correlation score indicative of the validity of the blood pressure signal is determined by:
pairing and comparing one or more features of the signals obtained from the pressure sensor and one or more features of the signals obtained from the pulse-related sensors; and
determining an agreement value between pairs of signals, wherein the correlation score is determined based on the agreement value.

According to some embodiments, the agreement value may be determined based on metrics applied to each of the signal pairs, or on a plurality of signal pairs over a designated time frame.

According to some embodiments, the pulse related sensor may include ECG, PPG, ICG, phonocardiography sensor, or any combinations thereof.

According to some embodiments, the motion sensor may include an accelerometer, a gyroscope, a magnetometer, an Inertial measurement Unit (IMU) or any combination thereof.

According to some embodiments, the pulse related signal may be selected from: continues pulse rate, heart rate, onset of pulse beat(s), separation of pulse beat(s) to segments, blood flow velocity.

According to some embodiments, the blood pressure signal may be associated with a blood pressure waveform.

According to some embodiments, there is provided a device for continuous, non-invasive blood pressure measurement of an ambulatory subject, the device includes:
a wearable body comprising a pressure sensor (or an array of pressure sensors) configured to be worn by the subject;
a pulse-related sensor associated with the wearable body; and
a processor configured to execute the method for continuous, non-invasive blood pressure measurement of subject while being active.

According to some embodiments, the device may further include a motion related sensor(s).

According to some embodiments, the pulse-related sensor may be comprised within the wearable body. According to some embodiments, the pulse-related sensor may be functionally associated with the wearable body.

According to some embodiments, there is provided a method for determining quality or validity of a blood pressure signal from a pressure sensor, obtained from a non-static subject, the method comprising:
receiving at least one blood pressure related signal from at least one pressure sensor;
receiving at least one pulse related signal from at least one pulse sensor;
calculating a correlation score between the blood pressure signal and the pulse related signal, wherein a correlation score above a predetermined threshold is indicative of the quality and/or validity of said blood pressure signal.

According to some embodiments, there is provided a method for continuous, non-invasive blood pressure measurement of a non-static subject, the method comprising: receiving at least one blood pressure related signal from at least one pressure sensor;
receiving at least one pulse related signal from at least one pulse sensor;
calculating a quality score based on integration of data derived from the pulse related signal and data derived from the blood pressure related signal, said quality score is indicative of quality of the blood pressure related signal; and
calculating blood pressure based on combined data obtained from the pressure sensor and the pulse sensor, if the quality score is above a predetermined threshold.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not drawn to scale. Moreover, two different objects in the same figure may be drawn to different scales. In particular, the scale of some objects may be greatly exaggerated as compared to other objects in the same figure.

In block diagrams and flowcharts, optional elements/components and optional stages may be included within dashed boxes.

In the figures:

FIG. 1A and FIG. 1B show isometric and side view schematic illustrations of a wearable device for non-invasive continuous blood pressure measurement while the subject is non-static, according to some embodiments;

FIG. 2 shows a general workflow diagram for sensor fusion method for non-invasive continuous blood pressure measurement, according to some embodiments;

FIG. 3 shows a workflow diagram for sensor fusion method for non-invasive continuous blood pressure measurement, according to some embodiments;

FIG. 4 shows exemplary determination of heartbeat onsets and adjustment thereof, using sensor fusion method, according to some embodiments. Top panel (a)—graph of pressure signals obtained over a period of time from a pressure sensor, indicating detected/identified heartbeat onsets (marked by dots); middle panel (b)—graph of synced signals obtained over a period of time from a pulse rate sensor (in this case PPG sensor), indicating detected/identified heartbeat onsets (marked by dots). Bottom panel (c)—graph of signals from the pressure sensor with modified onset detection, based on matching between the information in Panels a and b;

FIG. 5 shows a workflow diagram for sensor fusion method for non-invasive continuous blood pressure measurement, according to some embodiments; and

FIG. 6 shows exemplary determination of heartbeat onsets based on information from pulse related sensor(s) and adjustment thereof, according to some embodiments. Top panel (a)—graph of pressure signals obtained over a period of time from a pressure sensor, indicating detected/identified heartbeat onsets (marked by dots); middle panel (b)—graph of synced signals obtained over a period of time from a pulse rate sensor (PPG), indicating detected/identified heartbeat onsets (marked by dots). Bottom panel (c)—graph of signals from the pressure sensor with modified onset detection, based on transposing information between Panels a and b.

### DETAILED DESCRIPTION

The principles, uses and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

In the following description, various aspects of the invention will be described. For the purpose of explanation, specific details are set forth in order to provide a thorough understanding of the invention. However, it will also be apparent to one skilled in the art that the invention may be practiced without specific details being presented herein. Furthermore, well-known features may be omitted or simplified in order not to obscure the invention.

According to some embodiments, there is provided a device for non-invasive continuous blood pressure measurement while the subject is non-static. According to some embodiments, the device may be a wearable device which may be capable of at least measuring pressure waveforms from the wrist or other body part of a subject wearing the device. According to some embodiments, the device may include a wearable body including at least a pressure sensor array and configured to be worn by a subject at a respective body part, such as wrist, arm, leg, ankle. According to some embodiments, the device may include a processor in communication with a non-transitory computer-readable storage medium, the storage medium has stored thereon one or more program codes (or one or more algorithms). In some embodiments, the algorithms facilitate integration of information/data from various sensors, to allow for motion compensation. According to some embodiments, the device may further include, or be associated with one or more motion sensors and/or pulse signal sensors (also referred to as pulse related sensors), such as, ECG, PPG, ICG, phonocardiography, to allow identification of motion induced artifacts, to thereby enhance accuracy and efficacy of blood pressure signals and measurements, in particular, when the subject is non-static.

According to some embodiments, the one or more algorithms may be configured to receive one or more signals from the various sensors, analyze that information to identify motion related events, and provide an accurate blood pressure calculation, while taking into account the motion related events.

According to some embodiments there are thus provided devices and methods for non-invasive continuous blood pressure measurement while the subject is non-static, utilizing sensor-fusion methods, whereby, information obtained from various sensors is integrated/analyzed/fused, to identify motion-related events, which may affect the blood pressure measurements, and to correct/adjust the measurements accordingly, to thereby provide improved and more accurate blood pressure readings. According to some embodiments, the device may be configured to acquire a continuous non-invasive arterial (such as, e.g., radial) pressure signal (or in other words, a signal associated with the blood pressure, such as, e.g., in the form of a pressure waveform) and one or more additional signals. According to some embodiments, the device and/or method disclosed herein may enable acquisition of the pressure waveforms (for example, in the format of a continuous pressure signal) prior to, concomitantly with, and/or after measurements from one or more additional one or more sensors, in particular, pulse related sensors and/or motion sensors. Accordingly, by utilizing blood pressure waveform signals and/or characteristics/parameters related thereto together with signals from pulse related sensors and/or additional motion related sensors, the devices and methods disclosed herein may thus allow a much more accurate identification of blood pressure signals, analyzing/identifying motion related artifacts/reading, to accordingly adjust the blood pressure readings.

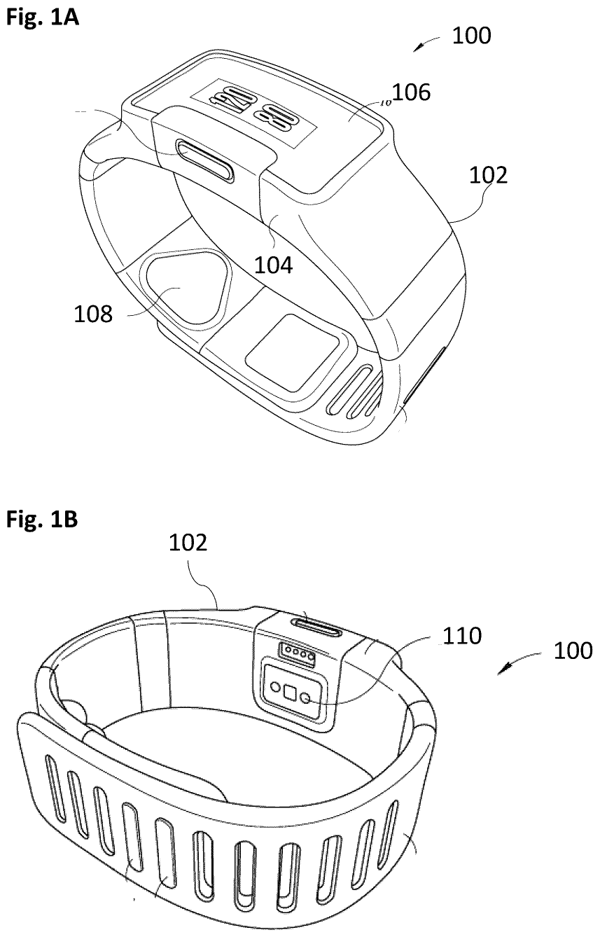

Reference is made to FIG. 1A and FIG. 1B, which show isometric and side view schematic illustrations of a device for non-invasive continuous blood pressure measurement while the subject is non-static, according to some embodiments.

According to some embodiments, device 100 may include a wearable body 102. According to some embodiments, the wearable body 102 may include a display 106 (such as, for example, a viewable OLED screen, etc.), which may be mounted in a housing 104. According to some embodiments, the wearable body 102 and/or the housing 104 may include a processor (for example, a CPU or MPU) and a storage module in communication therewith. According to some embodiments, the communication between the processor and the storage module may be wired and/or wireless. According to some embodiments, the wearable body may include any one or more of: buttons, switches or dials, touch pad or screen, a band (and/or one or more straps), and/or a fastening mechanism configured to fasten the wearable body to the subject. According to some embodiments, the wearable body may include one or more pressure sensors (for example, in the form of an array), configured to sense pressure of an artery such as the radial and/or ulnar arteries.

According to some embodiments, the wearable body 102 may include a sensor array 108 configured to sense the pressure waveform from one or more blood vessels of the subject. According to some embodiments, the sensor array 108, which may include one or more pressure sensors, is positioned such that the one or more pressure sensors are positioned against the wrist of the subject. According to some embodiments, when the wearable body 102 is fastened to the subject, the sensor array 108 may be positioned on (or near) at least one of the radial, ulnar and brachial arteries. According to some embodiments, the wearable body 102 and/or the sensor array 108 may be configured to apply medium pressure to any one or more of the radial, ulnar and brachial arteries (i.e., for example, a pressure that is significantly less than the systolic pressure but enough to sense the pressure waveform). According to some embodiments, the wearable body 102 may include one or more additional sensors 110 such as, for example, an optical sensor, a pulse related sensor, a motion sensor, and the like. In some embodiments, the one or more additional sensors may be selected from: accelerometer, gyroscope, magnetometer, Inertial Measurement Unit (IMU), ECG, PPG, ICG, phonocardiography, or any combination thereof.

According to some embodiments, the methods for enabling non static continuous vital signs monitoring utilizing wearable devices having pressure sensors overcome motion related artifacts and can thus enable continuous measurement, incorporate and integrate information from additional sensors (such as, pulse related sensors), which are more robust to motion. In some embodiments, such sensor-integration (also referred to as sensor fusion) methods may include one or more steps of: signal correlation, signal validation, and/or heartbeat detection.

In some embodiments, when utilizing signal validation, the blood pressure calculation uses pressure waveforms, but also uses beat detection based on the pulse-related signal, and only when both the pressure waveform and the pulse-related signal detections agree on the same beats, blood pressure (BP) calculation are applied on those beats. When the subject is non-static, the pulse-related signal and optionally information from a motion sensor (such as, accelerometer, gyroscope, IMU (Inertial Measurement Unit)) is used to identify heartbeats for validation of beats that has been detected/sensed by the pressure sensors, for the determination/calculation of the blood pressure.

In some embodiments, when utilizing heartbeat detection—the beat detection is facilitated using the pulse-related signal, and the blood pressure calculation is performed on those beats. When the subject is non-static, the pulse-related signal and optionally information from a motion sensor is used to identify heartbeat segments, which can be thereafter segmented from the pressure sensors signals, for more accurate blood pressure calculation.

According to some embodiments, there is provided a wearable blood pressure and vital signs monitoring device. In some embodiments, the device may include a plurality of sensors and a processing unit configured to apply a method for sensors' data fusion enabling static and non-static continuous monitoring of vital signs, providing accurate vital signs measurements while the subject is non-static, e.g., during activity. In some embodiments, the device includes a plurality of sensors, including at least one pressure sensor (for example, in the form of a sensor array), at least one sensor capable of measuring cardiovascular physiological signals and/or at least one motion related sensor. In some embodiments, as detailed below herein, the device utilizes sensor fusion methods (algorithms) to overcome motion related artifacts to enable continuous measurement, by incorporating data from the various sensors.

In some embodiments, the disclosed system, device and method allow the fusion of information from the pressure sensors with the output of motion related ones and any/or a combination of other vital signal sensors capable of allowing pulse detection is possible. The subsequent signal processing facilitates the mitigation of the motion artifacts using the information from the additional sensors, while having the pressure wave signals (obtained from the pressure sensors) as the base and reference signal for the blood pressure calculation/determination.

According to some embodiments, the wearable device disclosed herein thus utilize the one or more pressure sensors to derive the blood pressure calculation. In some embodiments, the device may further use one or more motion sensing sensors, together with the one or more pressure sensors to apply an algorithm, based on data received from these sensors, for motion cancelation or motion compensation, to thereby reduce motion artifact and improve signal quality. In some embodiments, processing and fusion of one or more pressure sensors respectively may allow motion artifact removal and correct pulse detection possible, by having the motion sensed signal as reference for motion artifacts identification and proper beat signal filtering, either in the time or frequency domain. Posterior distinction between pulse and artifact signals may be accomplished by, but not limited to, the autocorrelation function or the magnitude spectrogram of different frequency bins.

In some embodiments, the wearable device disclosed herein may further utilize data from one or more additional physiological sensor/sensor system capable of acquiring and measuring at least one physiological feature from an artery. Such sensors may be functionally and/or physically associated with the device. In some embodiments, the additional sensor can be one that is unaffected by motion (for example, ECG patches) or mostly unaffected (for example, phonocardiography microphones). Additional or alternatively, the device may include or be associated with a sensor system including a physiological sensor (e.g., PPG) together with motion sensing sensors (such as accelerometer or gyroscope) and may further utilize a robust method (algorithm) for motion cancelation, based on data obtained from the sensors. In some embodiments, the additional sensor or sensor system is able to detect one or more physiological features related to the hemodynamic system which may include the following: continuous pulse rate (heart rate), onsets of pulse (heart) beats or other separation of the signal to specific pulses, R-R intervals, and the like, or any combination thereof. In some embodiments, it may further include detection of periods of time where motion artifacts are too severe to be able to extract adequate quality signal and derive accurate features.

According to some embodiments, when referring to physiological signal or physiological sensor system that requires motion cancelation, the signal may be processed for motion compensation. In some embodiments the pressure signal may be initially preprocessed using motion cancelation or motion compensation algorithms. In some embodiments, the signal may be used without such a preprocessing step.

According to some embodiments, for a sensor-fusion based method as disclosed herein, the heartbeats separation process may be based on the pressure sensors signal but it is further validated by an additional heartbeat detection and separation performed using any type of signal directly related to the circulatory system (e.g., ECG, PPG, RF based), and possibly the fusion of more than one signal. The reference signal is either insusceptible to motion or allow integration of motion compensation techniques (e.g., using accelerometer, accelerometer, gyroscope, magnetometer, Inertial measurement Unit (IMU), and the like, or any combination thereof).

According to some embodiments, the sensor fusion method (algorithm) may utilize signal correlation steps for pressure sensors signal quality assessment. While motion is detected, the device may acquire both the pressure sensor signals and information from the one or more additional pulse related sensors. In some embodiments the pressure signal may be initially preprocessed and/or filtered using motion cancelation or motion compensation algorithms by using the motion sensed signal as noise reference to be removed. In some embodiments, a synchronization step may be applied to match the signals from the different sensors. In some embodiments, the synchronization step may be unnecessary, if, for example, the signals are obtained from the same artery, at the same location. Thereafter, the algorithm may include a step of evaluation of a correlation score, utilizing, for example, a correlation function between the synchronized signals to assess the pressure signals quality. A strong correlation indicates a suitable signal quality, while low correlation suggests that the signal may not be suitable for blood pressure calculation.

In some exemplary embodiments, pulse sensors (such as, for example, PPG or ECG) may be used as assistance to ensure accurate measurement. In such examples, PPG/ECG (or any other relevant sensor, such as, PPT, PWV, ICG) information may be used for “Quality Verification” purposes, whereby the device calculates signal correlation of PPG/ECG signals and pressure sensors signal (over a relatively short/small time window), if the correlation is high then BP calculation is applied using the pressure sensor data of that time window. When the wearable device is in motion (i.e., the subject is non-static), then the PPG/ECG signal may use motion cancelation filtering together with one or more motion related sensors such as: accelerometer, gyroscope, magnetometer, or IMU. In some embodiments, the pressure sensor may also use motion cancelation filtering with motion related sensor.

In some embodiments, the correlation function may include such functions as, but not limited to: cross correlation, cross spectra density, coherence or mean squared coherence (MSC), and the like, or any combination thereof. In some embodiments, the correlation function may be applied to the obtained signals, derivatives of the signal (i.e., data derived from, or data related to), or any other transformation of the signals. In some embodiments, when a strong correlation of all sensors is identified, then the relevant signal segment is considered to be validated. These validated pressure signal segments then become the raw signal input for a blood pressure calculation algorithm.

In some embodiments, the correlation may also be carried out in the frequency domain using techniques such as, but not limited to: cross spectrum analysis, power spectrum similarity, and the like, or combinations thereof.

According to some embodiments, the sensor fusion algorithm may thus improve the quality of the BP calculations.

According to further embodiments, the sensor fusion method disclosed herein can improve accuracy of BP calculations using the wearable device. In such embodiments, the sensor fusion method is used to apply signal (quality) validation steps for the pressure sensors signal. When motion is detected, the device can utilize information from the additional pulse-related sensor to calculate one or more physiological features (for example, pulse rate or pulse onsets). The device may also utilize the pressure sensor to acquire pressure waveforms, and calculate corresponding physiological feature(s). Then, a step of sensor fusion method is applied, where the features from the pressure sensor and the physiological sensor are paired and compared. In some embodiments, the pressure signal and the physiological sensor signals may be synchronized prior to pairing the features. Next, the features of all sensors are tested for agreement, using various metrics (such as, for example, distance or error (relative or absolute)), which can be applied to each pair and/or on a specific time window, that may include more than a pair (i.e. a plurality of pairs). The agreement test may require that the metric is below or above specific values, or within a specific range of values. When the features of all sensors agree, the relevant signal segment is determined as “validated”. These validated pressure signal segments in turn can become the raw signal input for a blood pressure calculation algorithm.

Thus, in some exemplary embodiments, pulse sensors (such as, for example, PPG, ECG) can be used as assistance to ensure accurate measurement. In such embodiments, PPG/ECG (or any other relevant sensor, such as, PPT, PWV, ICG) information may be used for “Validation” purposes, whereby the device performs beat detection with both PPG/ECG and the pressure sensors. If both types of sensors (i.e., pulse related sensor and pressure sensor), agree on the beats, then BP calculation may be applied using the pressure sensor data of those beats. When the device is in motion, then using PPG/ECG beat detection can also be carried out together with one or more of motion related sensors (such as accelerometer, gyroscope, magnetometer, or IMU), to identify heartbeat(s) for validation of beats detected by pressure sensors for BP calculation.

Thus, according to some embodiments, the sensor fusion algorithm can perform signal validation, whereby the blood pressure calculation utilizes the wearable device's pressure sensors to acquire pressure waveforms, and also utilizes beat detection based on the pulse-related signal. Only when beat detections from both pressure waveform and pulse-related signal agree on the same beats then BP calculation of those beats is applied. When the user is non-static, the algorithm can used information from one or more motion related sensors to apply motion compensation filter to the pulse-related signal, and identify heartbeats on for validation of beats detected by pressure sensors for BP calculation. Furthermore, when the user is non-static, the algorithm may use data from one or more motion related sensors (such as, accelerometer, gyroscope, magnetometer, Inertial measurement Unit (IMU)), to apply motion compensation filter to the pressure signal as well. These can be the same sensors used to determine the pulse-related signal or additional sensor(s), each one situated in close proximity to the corresponding respective sensors (i.e., pulse-related signal sensors or pressure sensors).

According to some embodiments, the validation or estimation of the accuracy of heartbeat identification can also make use of computation of heart rate or pulse duration. In such embodiment, the heart rate or pulse duration is calculated using one or more reference signals (e.g., PPG, ECG) that aid in the beat extraction process in a secondary signal (pressure signal), in which the starting and ending points of the pulses may be identified inaccurately due to motion, but where the secondary signal is more suitable for BP calculation. Ideally, the heart rate related frequencies from the reference signal determine the filtration of the pressure signal and approval of the heartbeat detections.

In some embodiments, the one or more reference signals are also used for heartbeat isolation. Then using sensor fusion techniques (such as, for example, but not limited to: a majority-vote, Borda count) segments with high certainty of accurate beat extraction and adequate signal quality can be identified to consequently improve the accuracy of the BP calculation when performed only in the selected segments.

In some embodiments, the motion cancelation filtering may be applied on the pressure signal as well to improve the signal quality, before evaluating the heartbeats compared to the reference signal.

In some embodiments, when Systolic (SYS) and Diastolic (DIA) blood pressure values are calculated based on several heartbeats, the reference signal may be used to assign/calculate an accuracy score (or weight) to each heartbeat. The resulting SYS and DIA values are calculated using the score—for example using a weighted average (instead of average over validated beats), or weighted cumulative moving average. Accuracy score is a measure of agreement between the heartbeats calculated from the reference signal and the pressure waveform and can be calculated using various methods for defining agreement and metrics. For example, as each heartbeat is defined by its beginning and end point (also referred to herein as “onsets”), an accuracy score per heartbeat (“Accₙ”) may be based on the distance (error) between the onsets extracted from the reference signal and the pressure waveform, divided by the heartbeat duration:

$${Acc}_{n} = 1 - \frac{\vert {P}_{n} - {\tilde{P}}_{n} \vert + \vert {P}_{n - 1} - {\tilde{P}}_{n - 1} \vert}{{P}_{n} - {P}_{n - 1}}$$

Where Pₙ is the location (in time) of onset n for the pulse related sensor and \tilde{P}ₙ is the location (in time) of onset n for the pressure sensor.

Another example of a possible accuracy score per heartbeat (“Accₙ”) is the normalized cross correlation between the signals at each heartbeat

$${Acc}_{n} = \frac{1}{N} \frac{{\sum }_{x} ( S {1}_{x} - \overline{S 1} ) ( S {2}_{x} - \overline{S 2} )}{\sqrt{{\sum }_{x} {( S {1}_{x} - \overline{S 1} )}^{2} * {\sum }_{x} {( S {2}_{x} - \overline{S 2} )}^{2}}}$$

Where S1 is Signal values of the pulse related sensor and S2 is Signal values of the pressure sensor.

In the case of a non-static measurement, using motion related sensors can improve the beat extraction process by providing additional information, allowing motion artifact cancellation. Motion compensation or motion artifact cancellation filters applied to any reference signal related to the circulatory system, can significantly improve the physiological signal quality prior to heartbeat isolation, thus increasing the overall accuracy.

According to some embodiments, preprocessing the pressure signals and/or the pulse related signals may include heartbeat segmentation (also referred to as “pulse detection”). According to some embodiments, heartbeat segmentation may include dividing at least a portion of the signals into a plurality of segments. According to some embodiments, each segment includes a heart cycle (and/or data associated with a heart cycle). According to some embodiments, the plurality of segments may be equivalent sets (or subset) of pulses (or heartbeats). According to some embodiments, equivalent sets (or subsets) may include the same number of sets (or subset) of pulses (or heartbeats). According to some embodiments, equivalent sets (or subsets) may include the about same number of sets (or subset) of pulses (or heartbeats). In some embodiments, the preprocessing may include at least one heartbeat normalization method, normalizing the heartbeats between different signals.

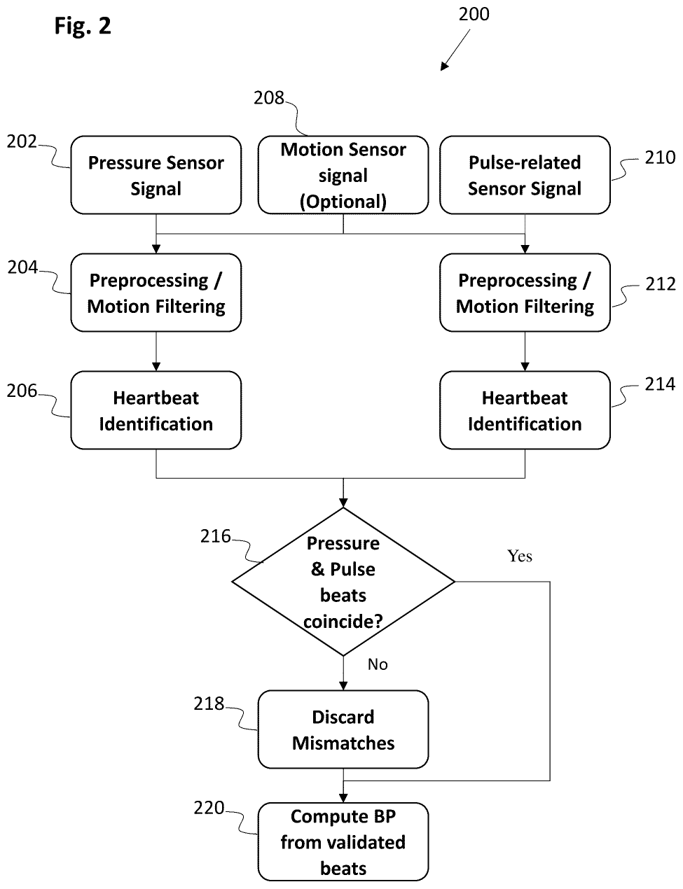

Reference is now made to FIG. 2, which shows a general workflow diagram for sensor fusion method for non-invasive continuous blood pressure measurement, according to some embodiments. As shown in FIG. 2, pressure sensor signal (202) is obtained (for a pressure sensor of a wearable device), optionally preprocessed and/or filtered (204), by applying various filters and calculations on the obtained pressure waveforms. The data derived from the waveform (parameters) is used to identify heartbeat (206). Further optional information from a motion sensor (208) may be obtained. In some embodiments, the preprocessing of the pressure sensor signal (204) may include motion compensation filtering using a motion sensor signal (208). Such information may be used in various calculations and preprocessing steps on information obtained from various sensors. In addition, information (signals) is obtained from one or more pulse related sensors (210) that may be physically and/or functionally associated with the wearable device. In some embodiments, the pulse related sensor may be housed within the wearable device. In some embodiments, the pulse related sensor may be connected to the wearable device (for example, by wires or wirelessly). In some embodiments, the pulse related sensors may include, for example, ECG sensor (for example, in the form of patches), PPG sensor, ICT sensor, phonocardiography sensor, and the like. In some embodiments, the pulse related sensor signal may be preprocessed and/or filtered (212), by applying various filters and calculations on the obtained signal, including, for example, filtering motion events (based, for example, on motion sensor signal (208). In some embodiments, the preprocessing of the pulse related sensor signal (212) may include motion compensation filtering using a motion sensor signal (208). The data derived from the pulse related signal (parameters) is used to identify heartbeat (214). At the next step, 216, the method compares/determines if the heartbeat information as determined based on the pressure sensor signal and the heartbeat as determined based on the pulse-related sensor signal coincide. If the two identified sets of heartbeats are determined to coincide, it is indicative that the measurement by the pressure sensor is valid/accurate and can be used to compute blood pressure (220), based on the information from the pressure sensor. If the two identified set of heartbeats are determined not to coincide, it is indicative that the measurement by the pressure sensor is not valid/not accurate, and is thus discarded (218), and is consequently not used for the computation of the blood pressure (220). In such instance, the measurement by the pressure sensor is considered an artifact (in particular, an artifact induced by motion).

Thus, in some embodiments, as shown in FIG. 2, when utilizing signal validation 200, the blood pressure calculation uses pressure waveforms 202, but also uses beat detection based on the pulse-related signal, and only when both the pressure waveform and the pulse-related signal detections (206 and 214 respectively) agree on the same beats 216, blood pressure (BP) calculation are applied on those beats 220. When the subject is non-static, the pulse-related signal 210 and optionally information from a motion sensor 208 (such as, accelerometer, gyroscope, magnetometer, IMU (Inertial Measurement Unit)) is used to identify heartbeats 214 for validation of beats that has been detected/sensed by the pressure sensors, for the determination/calculation of the blood pressure. When motion is detected and prior to heartbeat identification, pressure waveforms can be also subjected to preprocessing and motion filtering techniques 204 by optionally using the information provided by the motion sensor 208.

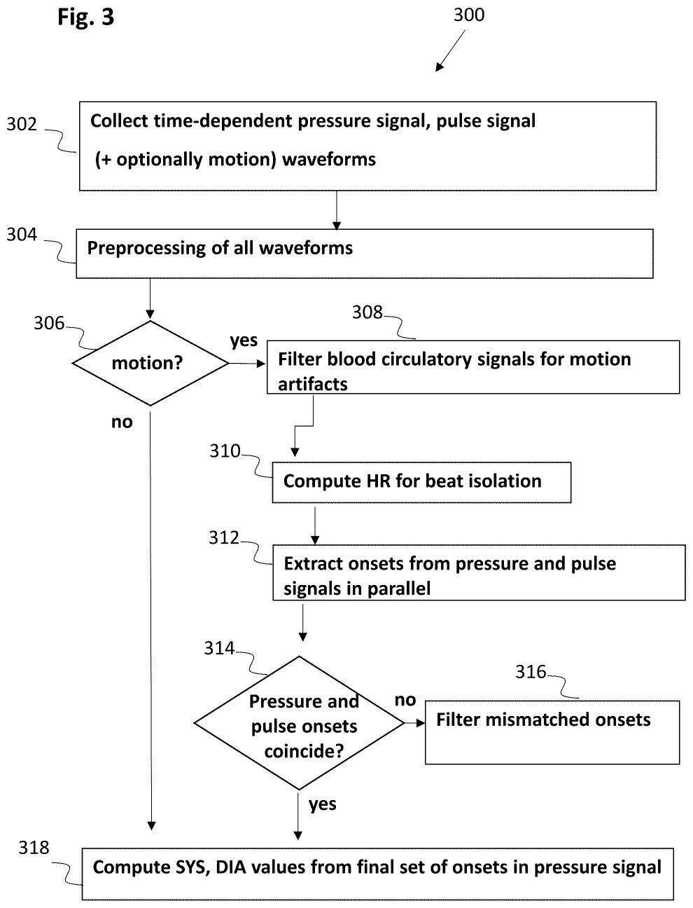

Reference is now made to FIG. 3, which shows a workflow diagram for sensor fusion method for non-invasive continuous blood pressure measurement, according to some embodiments. As shown in FIG. 3, method 300 for sensor fusion includes at step 302 the collection/obtaining of time-dependent signals (in particular, waveforms), including pressure signals (obtained from a pressure sensor of a wearable device), pulse-related signals (obtained from one or more pulse related sensors connected to or associated with the wearable device), and motion related signals (obtained from motion related sensor). At step 304, the obtained signals (waveforms) are preprocessed, by applying various filtering and calculations, as needed. At step 306 it is determined if motion is detected (for example, based on information from the motion sensor and/or information from the pulse-related sensor). In case motion is detected, at step 308, signals from the pulse related sensor (blood circulatory signals) are filtered to remove motion artifacts, and heart rate is computed at step 310, to isolate distinct heartbeats (beat isolation). In case motion is not detected at step 306, the information from the pulse related signals is used without filtering, for the computing of heart rate at step 310, to isolate distinct heartbeats. At step 312, the method includes extraction of onsets (i.e., identifying start and end point of heartbeats), from both the pressure related signals and the pulse-related signals. The extraction may be performed in parallel, to improved accuracy. Next, at step 314, it is determined if heartbeat information (in particular, the onsets of heartbeats) as determined based on the pressure sensor signal and the pulse-related sensor signal, coincide. If the onsets are determined to coincide, it is indicative that the measurement by the pressure sensor is valid/accurate, and a selected set of onsets (“final set of onsets”) in the pressure related signal can be used to compute various blood pressure values, such as, diastolic and systolic values. If the onsets are not determined to coincide, such mismatched onsets are discarded/filtered in step 316 and are consequently not part of the final set of onsets of the pressure sensor measurements and accordingly not used for the blood pressure values at step 318.

In some embodiments, as shown in FIG. 3, when utilizing heartbeat detection, the beat detection is facilitated using the pulse-related signal, and the blood pressure calculation is performed on those beats. When the subject is non-static 306, the pulse-related signal 302 and optionally information from a motion sensor 302 is used to identify heartbeat segments 310, which can be thereafter segmented from the pressure sensors signals 312, for more accurate blood pressure calculation.

As an example of a specific embodiment of the testing if pulse onsets coincide (314) may use two step method—first pairing between pulse related signal onsets and pressure signal onsets, followed by assessing the correspondence of the paired onsets. The pairing of onsets can be carried out by examining if the onset of the pressure signal (labeled as the pairing signal) is within a valid radius around an onset of the other pulse related signal (labeled as the reference signal). The pairing may result in one-to-many matching-one reference signal onset may be matched to many parings signal onset and vice versa. Alternative, the pairing may be one to one, where each onset is matched only to a single onset based on some distance metric. Next for each pair of matched onsets the coincidence (or mismatch) can be assessed.

In some embodiments the pairing of onsets can be carried out by searching for each onset from the pulse related signal (labeled as the reference signal), if pressure signal onset (labeled as the pairing signal) is within a valid range around the onset. In some embodiments a maximum range (Rmax) of action around the onset of the reference signal can be used. In some embodiments, this maximum range can be symmetric in time (Rmaxₜ[n−1]=Rmaxₜ[n+1]), or asymmetric-different for each side of the onset (prior time to preceding time (Rmaxₜ[n−1]≠Rmaxₜ[n+1]) as described in the following straightforward formula for valid range:

$$Range [ n ] = 〈 {TO}_{ref} [ n ] - R {max}_{t} [ n - 1 ] , {TO}_{ref} [ n ] + R {max}_{t} [ n + 1 ] 〉$$

where (TO_{ref}[n]) is the time of the reference onset n. The notion [n] denotes the pulse number n in the signal, [n−1] the previous pulse of the signal and [n+1] the following one.

Alternatively, other methods could be used to define the valid range, including but not limited to:

Simple symmetric pulse related range-calculating the previous pulse duration by means of the previous onset (TO_{ref}[n−1]) and using it together with a predefined ratio to (R_{%}) to calculate the range:

$$Range [ n ] = 〈 {TO}_{ref} [ n ] - {R}_{\%} * ( {TO}_{ref} [ n ] - {TO}_{ref} [ n - 1 ] ) , \\ {TO}_{ref} [ n ] + {R}_{\%} * ( {TO}_{ref} [ n ] - {TO}_{ref} [ n - 1 ] ) 〉$$

Or alternatively using the maximal range to bound the valid range:

$$Range [ n ] = 〈 {TO}_{ref} [ n ] - min ⌊ R max , {R}_{\%} * ( {TO}_{ref} [ n ] - {TO}_{ref} [ n - 1 ] ) ⌋ , \\ {TO}_{ref} [ n ] + min ⌊ R max , {R}_{\%} * ( {TO}_{ref} [ n ] - {TO}_{ref} [ n - 1 ] ) ⌋ 〉$$

Symmetric pulse related range-calculating the adjacent pulse duration by means of the previous (TO_{ref}[n−1]) and the following (TO_{ref}[n+1]) onsets with the onset in question (TO_{ref}[n]) and using it together with a predefined ratio (R_{%)} to calculate the range:

$$Range [ n ] = 〈 {TO}_{ref} [ n ] - {R}_{\%} * 0.5 * ( {TO}_{ref} [ n + 1 ] - {TO}_{ref} [ n - 1 ] ) , \\ {TO}_{ref} [ n ] + {R}_{\%} * 0.5 * ( {TO}_{ref} [ n + 1 ] - {TO}_{ref} [ n - 1 ] ) 〉$$

Or alternatively using the maximal range to bound the valid range:

$$Range [ \text{} n ] = \text{} 〈 {TO}_{ref} [ n ] - min ⌊ R max , {R}_{\%} * 0.5 * ( {TO}_{ref} [ n + 1 ] - {TO}_{ref} [ n - 1 ] ) ⌋ , {TO}_{ref} [ n ] + min ⌊ R max , {R}_{\%} * 0.5 * ( {TO}_{ref} [ n + 1 ] - {TO}_{ref} [ n - 1 ] ) ⌋ 〉$$

Non-Symmetric pulse related range-calculating the prior and preceding pulse durations by means of the previous (TO_{ref}[n−1]) and the following (TO_{ref}[n+1]) onsets with the onset in question (TO_{ref}[n]) and using it together with a predefined ratio to (R_{%)} to calculate the range:

$$Range [ n ] = 〈 {TO}_{ref} [ n ] - {R}_{\%} * ( {TO}_{ref} [ n ] - {TO}_{ref} [ n - 1 ] ) , \\ {TO}_{ref} [ n ] + {R}_{\%} * ( {TO}_{ref} [ n + 1 ] - {TO}_{ref} [ n ] ) 〉$$

Or alternatively using the maximal range to bound the valid range:

$$Range [ n ] = 〈 {TO}_{ref} [ n ] - min ⌊ R max [ n - 1 ] , {R}_{\%} * ( {TO}_{ref} [ n ] - {TO}_{ref} [ n - 1 ] ) ⌋ , \\ {TO}_{ref} [ n ] + min ⌊ R max [ n + 1 ] , {R}_{\%} * ( {TO}_{ref} [ n + 1 ] - {TO}_{ref} [ n ] ) ⌋ 〉$$

Once onset pairing step is done, various methods may be used to assess the coincidence (or mismatch) of each pair of onsets (314), including but not limited to:

Normalized time difference—the time difference between the onsets divided by (normalized) the pulse duration. The absolute difference in time between the pressure signal's onset and the pulse related signal's onset is divided by the pulse duration. The pulse duration may be calculated based on the pulse related signal duration:

$${T}_{diff} = \vert {TO}_{puls} [ n ] - {TO}_{pres} [ n ] \vert / ( {TO}_{puls} [ n ] - {TO}_{puls} [ n - 1 ] )$$

Or alternatively, average of the pressure and pulse related signal durations

$${T}_{diff} = \vert {TO}_{puls} [ n ] - {TO}_{pres} [ n ] \vert / \{ 0.5 * ( {TO}_{pres} [ n ] - {TO}_{pres} [ n - 1 ] ) + 0.5 * ( {TO}_{puls} [ n ] - {TO}_{puls} [ n - 1 ] ) \}$$

Where T_{diff} is the time difference assessment, TOₚᵣₑₛ[n] and TOₚᵤₗₛ[n] represent the time of onset n of the pressure signal and the pulse relate signal respectively. Thus, lower values of T_{diff} mean a better coincidence whereas higher values describe a greater mismatching. Therefore, if T_{diff} is below a specific threshold the onset would be considered as valid, otherwise it would be discarded.

Pulse matching ratio—the coincidence value can be calculated by analyzing the matching percentage between pulses duration of the pressure signal and pulse related signal pulses. Then, the matching ratio (match[n]) of the onset n is assessed by dividing the intersection of the pressure signal pulse interval (τₚᵣₑₛ[n]) and pulse time interval (τₚᵤₗₛ[n]) by the union of the same intervals. Where the time interval (τₚᵣₑₛ[n]) is defined as a fictitious pulses centered on the onsets of the pressure signal and the pulse-related one, i.e.:

$${τ}_{pres} [ n ] = 〈 \begin{matrix}{TO}_{pres} [ n - 1 ] + 0.5 \times ( {TO}_{pres} [ n ] - {TO}_{pres} [ n - 1 ] ) , \\ {TO}_{pres} [ n ] + 0.5 \times ( {TO}_{pres} [ n + 1 ] - {TO}_{pres} [ n - 1 ] )\end{matrix} 〉$$

Where the τₚᵣₑₛ[n] is the time range of the fictitious pulse of the pressure signal centered on the onset of pulse number n.

Accordingly, to the pulse-related signal:

$${τ}_{puls} [ n ] = 〈 \begin{matrix}{TO}_{puls} [ n - 1 ] + 0.5 \times ( {TO}_{puls} [ n ] - {TO}_{puls} [ n - 1 ] ) , \\ {TO}_{puls} [ n ] + 0.5 \times ( {TO}_{puls} [ n + 1 ] - {TO}_{puls} [ n - 1 ] )\end{matrix} 〉$$

And the matching ratio (match[n]) of the onset n is the intersection of the time intervals defined by τₚᵣₑₛ[n] and τₚᵤₗₛ[n] divided by the union of the same time intervals, i.e.:

$$match [ n ] = ( {τ}_{pres} [ n ] ⋂ {τ}_{puls} [ n ] ) / ( {τ}_{pres} [ n ] ⋃ {τ}_{puls} [ n ] )$$

The values of matching ratio (match[n]) are between 0 to 1, and lower values of matching ratio mean a greater mismatching whereas values closer to 1 describe a better coincidence of the onsets. Therefore, if match[n] is above a specific threshold the onset would be considered as valid, otherwise it would be discarded.

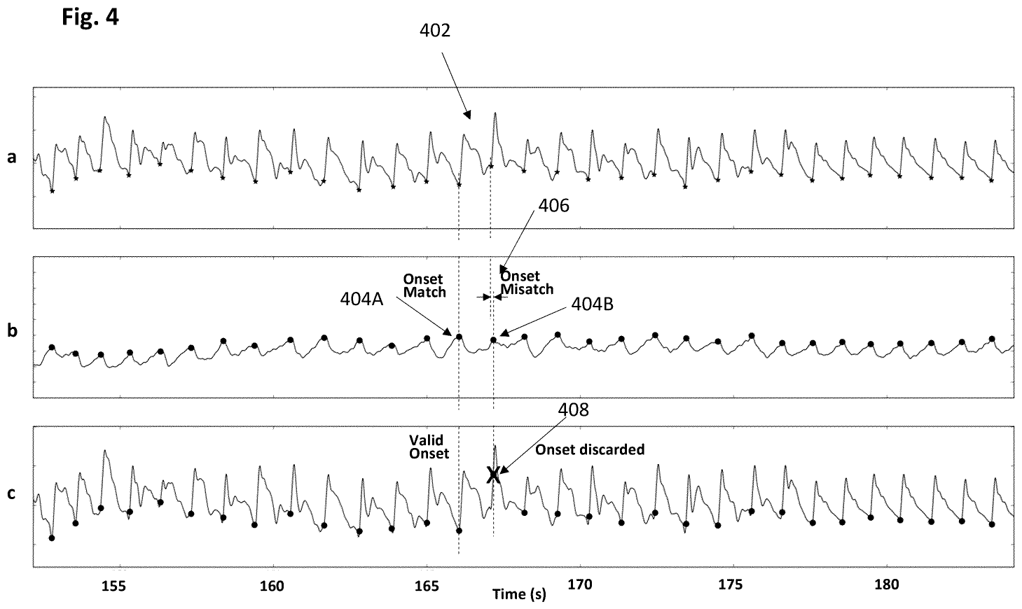

Reference is now made to FIG. 4, which shows exemplary determination of heartbeat onsets and adjustment thereof, using sensor fusion method, according to some embodiments. Shown in FIG. 4 are graphs of signals obtained over a period of time from a pressure sensor (top panel (a)) and a pulse rate sensor (PPG in this case, middle panel (b)). Heart beat onsets (marked by dots) are determined based on the pressure signal (top panel) and the PPG signals (panel b). The onsets determined based on either sensor information are compared/synchronized, to identify mismatches between each pair of onsets (for example, mismatches in the temporal appearance of onsets). The comparison/synchronization between the onsets may be performed over any desired time periods/time windows (for example over the entire time period of measurements, over interim time periods (which may be consecutive or intermittent), and the like). As shown in FIG. 4, at exemplary pulse 402, onset 404A, as determined based on the pressure sensor data and the pulse related sensor data matches, whereas onset 404B, a mismatch 406 is identified between the data obtained from the two sensors. Accordingly, as shown in bottom panel (panel c) of FIG. 4, based on the identified mismatch (which may be attributed to a motion artifact), the pressure sensor signal is adjusted, and the mismatched onset 408 is discarded and consequently not used for the blood pressure calculation.

According to further embodiments, the fusion sensor algorithm may utilize information from the additional sensors (i.e., the pulse related sensors and/or the motion sensors) as input from at least some of the processing steps of the blood pressure algorithm calculation, instead of the pressure sensor signals. When motion is detected, the device can acquire both the pressure sensor signals and the one or more additional sensors. In some embodiments the pressure signal may be initially preprocessed using motion cancelation or motion compensation algorithm. In some embodiments of this sensor fusion method, a synchronization step is applied to match the signals from different sensors. The synchronization step may be unnecessary if the signals are from the same artery at the same location. The device (in particular, the processing unit thereof) may use data from the additional sensor(s) to perform algorithmic steps, such as, for example, heart rate estimation and heartbeat onsets detection, instead of the pressure sensor data. The device may continue using blood pressure calculation process using the pressure sensor data on each beat, as segmented from the data obtained by the additional sensor. In some embodiments, this method can include additional step of evaluation of the pressure signal quality at each of the beats identified, and discarding poor quality signal, before the BP calculation.

In such embodiments, data from the pulse related sensors (with motion compensation) is used as main source of information for the heartbeat extraction step. Thereby assisting in ensuring accurate measurement. The pulse related sensors information is used for heartbeat detection, which may then be followed by BP calculation, using the pressure sensor data of those beats. As detailed above, an additional step of evaluation of the pressure signal quality at the identified beats, and discarding poor quality segments, before the BP calculation may be performed. When the device is in motion using the pulse related sensors beat detection can also be carried out together with one or more of motion related sensors, to identify heartbeats for validation of beats detected by the pressure sensors for the calculation of the blood pressure.

In some embodiments, the same blood circulatory condition may be measured in different underlying physiological techniques (e.g., pressure, PPG, ECG). While the physiological techniques or measured signal may vary, heartbeat isolation based on all these techniques inherently produce the same pulse identification (regardless the phase shift caused by the anatomy and physiological nature of the acquired signal).

In some embodiments, when computing blood pressure, fine or useful signal segments may be assessed depending on the certainty of extracting adequate quality pulses therefrom. In a noisy scenario, where motion artifacts can distort the signal, and motion artifact may be misidentified as heartbeats or pulses, having extra sources for ensuring a correct pulse identification may be vital. Thus, the wearable device is configured to extract heartbeats using one (or more) signal from one (or more) sensors to be used as reference for enhanced artifact pressure prone sensor signal, which is consequently used for the measurement and calculation of the blood pressure.

According to some embodiments, using the sensor fusion methods facilitates a robust (cleaner, less noisy) blood circulatory signal (i.e., pulse related signal (such as obtained from PPG or ECG) after (if needed) the application of motion compensation algorithms, can be used as a main source of information for the heartbeat extraction step, i.e., assisting in the assessment of the starting and ending points of each pulse. This segmentation by time to specific pulses segmentation may then transpose to the pressure signal data, being too noisy for accurately isolating beats directly therefrom, but while including the real information for blood pressure computation. In order to ensure the accuracy of the BP calculation, an additional step of evaluating the pressure signal quality at the identified beats, discarding poor quality pulses may be performed prior to the BP calculation.

In some embodiments, in a non-static scenario, sensors of movement can be integrated in the fusion-sensor system as an aid in the motion artifact cancellation process. Such noise filtering might be applied to the blood circulatory signals (pulse related signal), making them cleaner before extracting the pulses and/or computing the BP values.

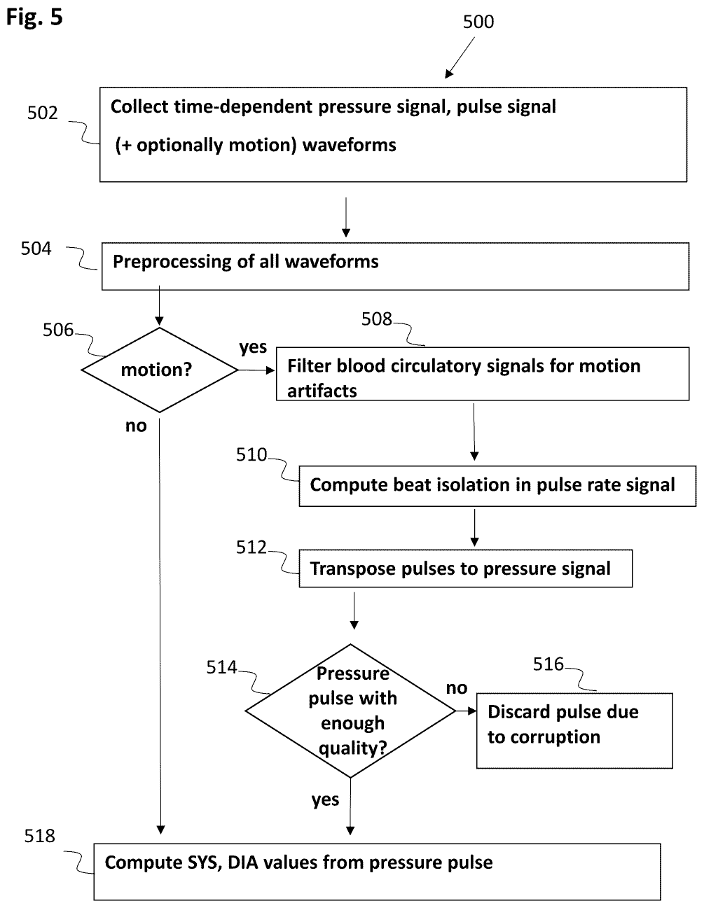

Reference is now made to FIG. 5, which shows a workflow diagram for sensor fusion method for non-invasive continuous blood pressure measurement, according to some embodiments. As shown in FIG. 5, method 500 for sensor fusion includes at step 502 the collection/obtaining of time-dependent signals (in particular, waveforms), including pressure signals (obtained from a pressure sensor of a wearable device), pulse-related signals (obtained from one or more pulse related sensors connected to or associated with the wearable device), and motion related signals (obtained from motion related sensor). At step 504, the obtained signals (waveforms) are preprocessed, by applying various filtering and calculations, as needed. At step 506 it is determined if motion is detected (for example, based on information from the motion sensor and/or information from the pulse-related sensor). In case motion is detected, at step 508, signals from the pulse related sensor (blood circulatory signals) are filtered to remove motion artifacts, and beat isolation (i.e., determination of heartbeat onsets) in the pulse rate signals are computed in step 510. In case motion is not detected at step 506, the information from the pulse related signals is used without filtering, for the computing of beat isolation is step 510, to identify onsets. At step 512, the method includes transposing the identified pulses (onsets) as determined based on the information from the pulse-related sensors, to the pressure signal, to allow downstream BP calculation based on the pressure sensor data at those transposed beats. At optional step 514 it is evaluated if the pressure signal quality at the identified beats is adequate, otherwise, low quality signals are discarded in step 516. In step 518, blood pressure values (such as, systolic and diastolic values) are computed based on the pressure sensor data, in particular data of the transposed beats (pulses). Thus, such method allows identifying valid/quality onsets, based on information from pulse rate sensors, and transposing said onsets to a corresponding pressure sensor data, to allow an accurate computation of blood pressure values, based on pressure sensor data obtained for the determined (validated/accurate) onsets.

Several methods can be combined to assess the quality of each individual pulse, including but not limited to:
Skewness, as a measure of the symmetry (or the lack of it) of the pulse x, which is defined as:

$${S}_{x} = \frac{1}{N} {\sum }_{i = 1}^{N} {( \frac{{x}_{i} - {μ}_{x}}{{σ}_{x}} )}^{3}$$

where μₓ and σₓ are the mean and standard deviation of x, respectively, and N is the number of samples in the pulse. To be considered as a pulse with enough quality, its skewness should be limited to a specific range of values.
Kurtosis, as a statistical measure used to describe the distribution of observed data around its mean. It represents a heavy tail and peakedness or a light tail and flatness of a distribution relative to the normal distribution, which is defined as:

$${K}_{x} = \frac{1}{N} {\sum }_{i = 1}^{N} {( \frac{{x}_{i} - {μ}_{x}}{{σ}_{x}} )}^{4}$$

where μₓ and σₓ are the mean and standard deviation of x, respectively, and N is the number of samples in the pulse. To be considered as a pulse with enough quality, its kurtosis should be limited to a specific range of values.

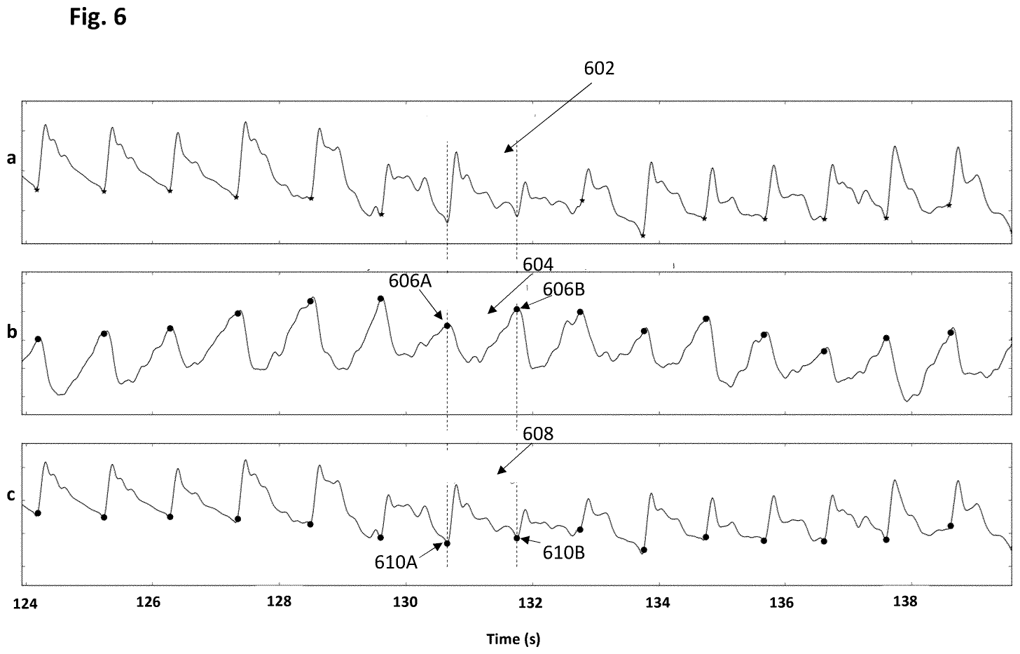

Reference is now made to FIG. 6, which shows exemplary determination of heart beat onsets based on information from pulse related sensor(s), in accordance with some embodiments. Shown in FIG. 6 are graphs of signals obtained over a period of time from a pressure sensor (top panel (a)) and a pulse rate sensor (PPG in this case, middle panel (b)). Heartbeat onsets (marked by dots) are determined based on the pressure signal (top panel) and the PPG signals (panel b). The data from the sensors is synchronized, to identify mismatches between each pair of detections. The comparison/synchronization between the onsets may be performed over any desired time periods/time windows (for example over the entire time period of measurements, over interim time periods (which may be consecutive or intermittent), and the like). As shown in FIG. 6, in panel (a), signal 602 of the pressure waveform, was not detected as pulse (i.e., no onsets were identified in the signal). In panel b, corresponding signal 604 was identified/isolated in the pulse related signal, having onsets 606A-B. Accordingly, as shown in bottom panel (c) of FIG. 6, based on the identified pulse based on the pulse sensor related signal, the pressure sensor signal is adjusted, and pulse 608 (with onsets 610A-B) is included with the pressure signal waveform and is used for the blood pressure calculation.

According to some embodiments, the pressure sensor may include any type of pressure sensor, such as a pressure sensor array, that may be included with/housed within the wearable device.

In some embodiments, the pulse related sensor may include any type of suitable sensor, including, for example, electrocardiogram (ECG), photoplethysmogram (PPG), Impedance cardiography (ICG), phonocardiography, and the like, or any combination thereof. Each possibility is a separate embodiment. The pulse related sensor may be attached to, associated with and/or integrated with the wearable device. In some embodiments, the motion sensor may be in communication with the processor of the wearable device and may be configured to send/receive information therefrom. In some exemplary embodiments, the pulse related sensor is PPG or ECG. The ECG is widely used clinically by placing up to 12 leads in the chest. Since this arrangement is not wearable, there are other techniques with enough signal to noise ratio (SNR) to be used in an ambulatory scenario. For example, wearable patches can record a single ECG channel with electrodes placed over the sternum or chest. Also, by integrating flexible textile electrodes into smart clothing kept tight against the user's body. The ECG electrodes can be also placed in a single armband or in combined positions along the arm for bipolar-ECG acquisition. In all the examples abovementioned, ECG signal may then be streamed either wired or wireless to the wearable device which can function as a data hub. In some embodiments, a technique suitable for punctual monitoring but with greater ECG signal quality consists of placing one electrode on one wrist, mounted together with the blood pressure sensors on the backside of wearable device. A second electrode placed on the front of the device, which may then be touched with the opposing hand, thereby creating the two sensing connection points needed on either side of the heart, to obtain a valid pulse related signal.

According to some embodiments, the motion related sensor may include any type of motion sensor, including, for example, accelerometer, gyroscope, magnetometer, Inertial measurement Unit (IMU), or any combination thereof. Each possibility is a separate embodiment. In some embodiments, the motion related sensor may be attached to, associated with and/or integrated with the wearable device. In some embodiments, the motion sensor may be in communication with the processor of the wearable device and may be configured to send/receive information therefrom. In some embodiments, motion compensation, based on information obtained from the motion sensor may be applied to pressure related data and/or pulse related data and may include, for example, using one or more adaptive filters.

It is understood that the term “one or more algorithms” may include a single algorithm or a plurality of algorithms. According to some embodiments, one algorithm may include therein a plurality of algorithms.

In the description and claims of the application, the words “include” and “have”, and forms thereof, are not limited to members in a list with which the words may be associated.

Unless otherwise defined, all technical and scientific terms used herein have the same meaning as commonly understood by one of ordinary skilled in the art to which this disclosure pertains. In case of conflict, the patent specification, including definitions, governs. As used herein, the indefinite articles “a” and “an” mean “at least one” or “one or more” unless the context clearly dictates otherwise.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although stages of methods according to some embodiments may be described in a specific sequence, methods of the disclosure may include some or all of the described stages carried out in a different order. A method of the disclosure may include a few of the stages described or all of the stages described. No particular stage in a disclosed method is to be considered an essential stage of that method, unless explicitly specified as such.

Although the disclosure is described in conjunction with specific embodiments thereof, it is evident that numerous alternatives, modifications, and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

The present invention may be a system, a method, and/or a computer program product. The computer program product may include a computer readable storage medium (or media) having computer readable program instructions thereon for causing a processor to carry out aspects of the present invention. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. Rather, the computer readable storage medium is a non-transient (i.e., not-volatile) medium.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may be executed entirely on the user's computer, partly on the user's computer, as a stand-alone software package, partly on the user's computer and partly on a remote computer or entirely on the remote computer or server. In the latter scenario, the remote computer (or cloud) may be connected to the user's computer through any type of network, including a local area network (LAN) or a wide area network (WAN), or the connection may be made to an external computer (for example, through the Internet using an Internet Service Provider) including wired or wireless connection (such as, for example, Wi-Fi, BT, mobile, and the like). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a wearable device, or other programmable data processing apparatus to produce a machine, such that the instructions, which execute via the processor of the device or other programmable data processing apparatus, create means for implementing the functions/acts specified in the flowchart and/or block diagram block or blocks. These computer readable program instructions may also be stored in a computer readable storage medium that can direct a device, a programmable data processing apparatus, to function in a particular manner, such that the computer readable storage medium having instructions stored therein includes an article of manufacture including instructions which implement aspects of the function/act specified in the flowchart and/or block diagram block or blocks.

The computer readable program instructions may also be loaded onto a wearable device, other programmable data processing apparatus, or other device to cause a series of operational steps to be performed on the computer, other programmable apparatus or other device to produce a computer implemented process, such that the instructions which are executed on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skilled in the art without departing from the scope and spirit of the described embodiments. The terminology used herein was chosen to best explain the principles of the embodiments, the practical application or technical improvement over technologies found in the marketplace, or to enable others of ordinary skilled in the art to understand the embodiments disclosed herein.

1. A method for continuous, non-invasive blood pressure measurement of subject while being active, the method comprising:
receiving at least one blood pressure related signal from at least one pressure sensor comprised in a wearable device;
receiving at least one pulse related signal from at least one pulse sensor;
calculating a correlation score between the blood pressure signal and the pulse related signal, said correlation score is indicative of quality and/or validity of the blood pressure signal; and
calculating blood pressure if the correlation score is above a predetermined threshold.

2. The method of claim 1, further comprising receiving a motion related signal from at least one motion sensor.

3. The method according to claim 1, further comprising a step of synchronizing the receiving of the blood pressure signal, the pulse-related signal and/or the motion related signal.

4. The method according to claim 1, further comprising preprocessing the blood pressure signal and/or the pulse related signal prior to calculating the correlation.

5. The method according to claim 4, wherein the preprocessing comprises one or more feature selection and/or onset detection in the blood pressure signal and/or the pulse related signal.

6. The method according to claim 4, wherein the preprocessing comprises motion cancelation or motion compensation algorithms applied to the blood pressure signal and/or the pulse related signal.

7. The method according to claim 1, wherein the correlation score is determined using a correlation function, configured to be applied to one or more of the obtained signals, or any parameters derived therefrom.

8. The method according to claim 1, wherein the correlation score indicative of the validity of the blood pressure signal is determined by:
pairing and comparing one or more features of the signals obtained from the pressure sensor and one or more features of the signals obtained from the pulse-related sensors; and
determining an agreement value between pairs of signals, wherein the correlation score is determined based on the agreement value.

9. The method according to claim 8, wherein the agreement value is determined based on metrics applied to each of the signal pairs, or on a plurality of signal pairs over a designated time frame.

10. The method according to claim 1, wherein the pulse related sensor comprises ECG, PPG, ICG, phonocardiography, or any combinations thereof.

11. The method according to claim 2, wherein the motion sensor comprises accelerometer, gyroscope, magnetometer, Inertial measurement Unit (IMU) or any combination thereof.

12. The method according to claim 1, wherein the pulse relate signal is selected from: continues pulse rate, heart rate, onset of pulse beat(s), separation of pulse beat(s) to segments, blood flow velocity.

13. The method according to claim 1, wherein the blood pressure signal is associated with a blood pressure waveform.

14. A device for continuous, non-invasive blood pressure measurement of an ambulatory subject, the device comprising:
a wearable body comprising a pressure sensor(s) configured to be worn by the subject;
a pulse-related sensor(s) associated with the wearable body; and
a processor configured to execute the method according to claim 1.

15. The device according to claim 14, further comprising a motion related sensor(s).

16. The device according to claim 14, wherein the pulse-related sensor is comprised within the wearable body.

17. The device according to claim 14, wherein the pulse-related sensor is functionally associated with the wearable body.

18. A method for determining quality or validity of a blood pressure signal from a pressure sensor, obtained from a non-static subject, the method comprising:
receiving at least one blood pressure related signal from at least one pressure sensor;
receiving at least one pulse related signal from at least one pulse sensor;
calculating a correlation score between the blood pressure signal and the pulse related signal, wherein a correlation score above a predetermined threshold is indicative of the quality and/or validity of said blood pressure signal.

19. A method for continuous, non-invasive blood pressure measurement of a non-static subject, the method comprising:
receiving at least one blood pressure related signal from at least one pressure sensor;
receiving at least one pulse related signal from at least one pulse sensor;
calculating a quality score based on integration of data derived from the pulse related signal and data derived from the blood pressure related signal, said quality score is indicative of quality of the blood pressure related signal; and
calculating blood pressure based on combined data obtained from the pressure sensor and the pulse sensor, if the quality score is above a predetermined threshold.
